(19) United States
(12) Patent Application Publication
(10) Pub. No.: US 2016/0331216 A1
(43) Pub. Date: Nov. 17, 2016

(54) ENDOSCOPE DEVICE

(71) Applicant: OLYMPUS CORPORATION, Tokyo (JP)

(72) Inventor: Yoshioki KANEKO, Tokyo (JP)

(73) Assignee: OLYMPUS CORPORATION, Tokyo (JP)

(21) Appl. No.: 15/218,250

(22) Filed: Jul. 25, 2016

Related U.S. Application Data
(63) Continuation of application No. PCT/JP2014/084122, filed on Dec. 24, 2014.

(30) Foreign Application Priority Data
Feb. 25, 2014 (JP) 2014-034639

(51) Int. Cl. A61B 1/045; A61B 1/018; A61B 1/00; A61B 1/005; A61B 1/05; A61B 1/06

(52) U.S. Cl. CPC A61B 1/045; A61B 1/05; A61B 1/0676; A61B 1/00009; A61B 1/00006; A61B 1/00045; A61B 1/0052; A61B 1/018; A61B 1/00018; A61B 1/00163

(57) ABSTRACT

An endoscope device includes: an insertion portion having an imaging optical system on a distal end of the insertion portion and configured to be inserted into a living body; a pressure detecting unit provided on the distal end of the insertion portion or ahead of the distal end and configured to detect contact with the living body by pressure; and an imaging unit configured to image an inside of the living body through the imaging optical system when a detection result of the pressure detecting unit satisfies a predetermined condition.

### CROSS REFERENCES TO RELATED APPLICATIONS

This application is a continuation of PCT international application Ser. No. PCT/JP2014/084122, filed on Dec. 24, 2014 which designates the United States, incorporated herein by reference, and which claims the benefit of priority from Japanese Patent Application No. 2014-034639, filed on Feb. 25, 2014, incorporated herein by reference.

### BACKGROUND

1. Technical Field

The disclosure relates to an endoscope device configured to be introduced into a living body to obtain information of the living body.

2. Related Art

Conventionally, endoscope devices have been widely used for various examinations in a medical field and an industrial field. A medical endoscope device can obtain an in-vivo image in a body cavity without cutting a subject by inserting an elongated flexible insertion portion, which has an image sensor with a plurality of pixels on a distal end of the insertion portion, into the subject such as a patient, so that a burden on the subject is reduced d such devices become prevalent (for example, refer to JP 2009-297428 A).

The endoscope device disclosed in JP 2009-297428 A is provided with an insertion portion having a stepped shape such that a distal end portion of which has a smaller diameter, the insertion portion including a contact detecting unit on a stepped portion of the stepped shape. In the endoscope device disclosed in JP 2009-297428 A, when a distal end of the insertion portion moves from a large diameter hole to a small diameter hole in the body cavity, the contact detecting unit detects contact with a stepped portion between the large diameter hole and the small diameter hole to determine that the insertion portion reaches the small diameter hole from the large diameter hole by the detection, and an imaging process and the like is performed based on the determination.

An observing system of the endoscope device includes obtaining the in-vivo image by allowing a distal end face of the endoscope to abut on a surface layer of the living body (for example, a surface of an organ). In the endoscope device, the distal end face is allowed to abut on the gland duct to obtain an image of the surface of the organ, for example. In the observing system, the image has be captured while at least the distal end face of the endoscope is in contact with the gland duct.

### SUMMARY

In some embodiments, an endoscope device includes: an insertion portion having an imaging optical system on a distal end of the insertion portion and configured to be inserted into a living body; a pressure detecting unit provided on the distal end of the insertion portion or ahead of the distal end and configured to detect contact with the living body by pressure; and an imaging unit configured to image an inside of the living body through the imaging optical system when a detection result of the pressure detecting unit satisfies a predetermined condition.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic view illustrating a configuration of an endoscope system according to a first embodiment of the present invention;

FIG. 2 is a schematic diagram illustrating the schematic configuration of the endoscope system according to the first embodiment of the present invention;

FIG. 3 is a schematic diagram illustrating a configuration of a distal end face of a surface layer observation endoscope according to the first embodiment of the present invention;

FIG. 4 is a flowchart illustrating a surface layer observing process performed by the endoscope system according to the first embodiment of the present invention;

FIG. 5 is a schematic view for illustrating an example of a configuration of a distal end of the surface layer observation endoscope according to the first embodiment of the present invention;

FIG. 6 is a schematic diagram illustrating: a configuration of a distal end of a surface layer observation endoscope according to a first modification of the first embodiment of the present invention;

FIG. 7 is a planar view in an arrow A direction in FIG. 6;

FIG. 8 is a flowchart illustrating a surface layer observing process performed by an endoscope system according to a second modification of the first embodiment of the present invention;

FIG. 9 is a flowchart illustrating a surface layer observing process performed by an endoscope system according to a third modification of the first embodiment of the present invention;

FIG. 10 is a schematic diagram illustrating a schematic configuration of an endoscope system according to a second embodiment of the present invention;

FIG. 11 is a schematic diagram illustrating a configuration of a distal end face of a surface layer observation endoscope according to the second embodiment of the present invention;

FIG. 12 is a flowchart illustrating a surface layer observing process performed by the endoscope system according to the second embodiment of the present invention;

FIG. 13 is a schematic diagram illustrating a schematic configuration of an endoscope system according to a third embodiment of the present invention;

FIG. 14 is a flowchart illustrating a surface layer observing process performed by the endoscope system according to the third embodiment of the present invention;

FIG. 15 is a schematic view illustrating an example of a posture estimation table used in an image obtaining process performed by the endoscope system according to the third embodiment of the present invention;

FIG. 16 is a flowchart illustrating a surface layer observing process performed by an endoscope system according to a fourth embodiment of the present invention;

FIG. 17 is a schematic view for illustrating a posture evaluation value calculating process performed by the endoscope system according to the fourth embodiment of the present invention;

FIG. 18 is a schematic diagram illustrating a configuration of a distal end face of a surface layer observation endoscope according to a modification of the fourth embodiment of the present invention;

FIG. 19 is a schematic view for illustrating a posture evaluation value calculating process performed by an endoscope system according to the modification of the fourth embodiment of the present invention;

FIG. 20 is a schematic diagram illustrating a schematic configuration of an endoscope system according to a fifth embodiment of the present invention;

FIG. 21 is a schematic diagram illustrating a schematic configuration of an endoscope system according to a modification of the fifth embodiment of the present invention; and

FIG. 22 is a schematic view for illustrating two-photon excitation fluorescence observation according to the modification of the fifth embodiment of the present invention.

### DETAILED DESCRIPTION

Modes for carrying out the present invention (hereinafter, referred to as “embodiment(s)”) are hereinafter described. In the following embodiments, reference will be made to an endoscope system provided with a medical endoscope device which captures an image in a subject such as a patient to display. The present invention is not limited by the embodiments. The same reference signs are used to designate the same elements throughout drawings.

### First Embodiment

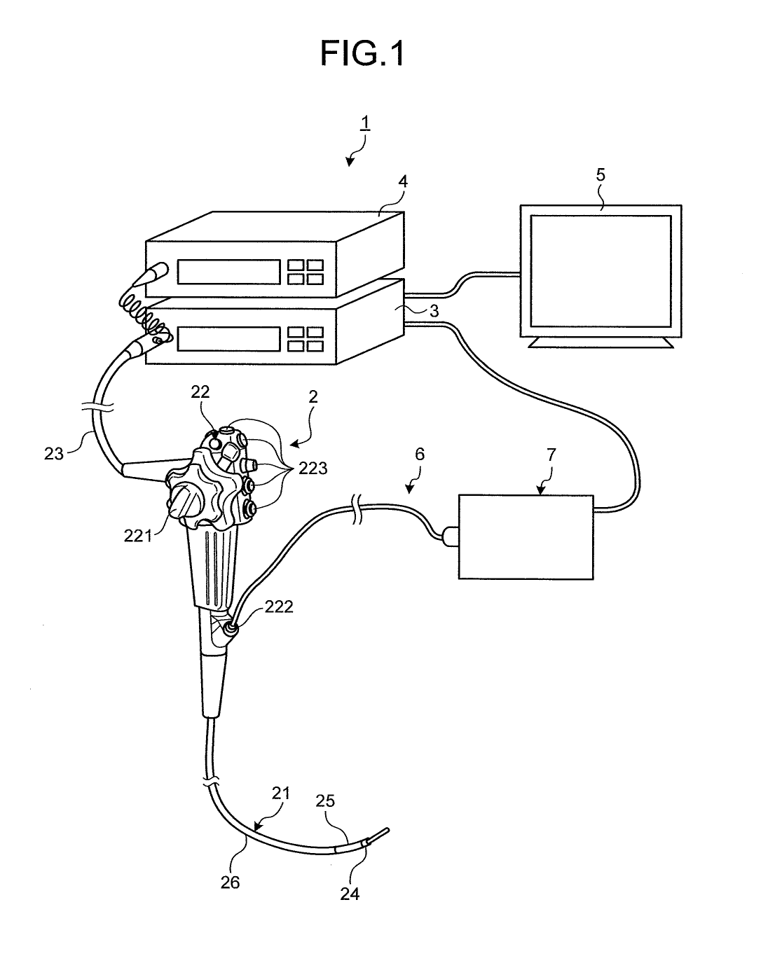

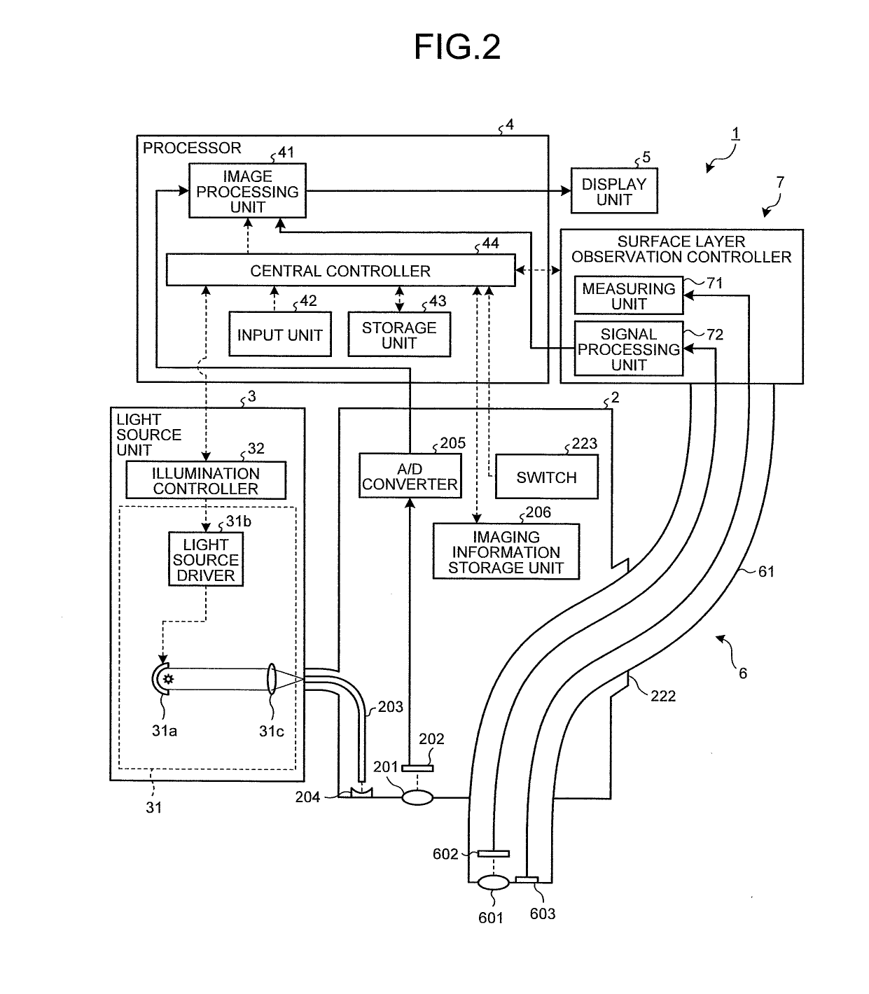

FIG. 1 is a schematic view illustrating a configuration of an endoscope system 1 according to a first embodiment of the present invention. FIG. 2 is a schematic diagram illustrating the schematic configuration of the endoscope system 1 according to the first embodiment The endoscope system 1 illustrated in FIGS. 1, and 2 is provided with a live observation endoscope 2 which captures an in-vivo image of an observed region to generate an electric signal with an insertion portion 21 inserted into a subject, a light source unit 3 which generates illumination light emitted from a distal end of the live observation endoscope 2, a processor 4 which performs predetermined image processing on the electric signal obtained by the endoscope and controls operation of an entire endoscope system 1, a display unit 5 which displays the in-vivo image after the image processing by the processor 4, a surface layer observation endoscope 6 configured to be inserted into the live observation endoscope 2 to capture an in-vivo image of the observed region while being in contact with the observed region (a surface layer of a living body) and to generate the electric signal, and a surface layer observation controller 7 which controls operation of an entire surface layer observation endoscope 6. The endoscope system 1 is configured to insert the insertion portion 21 into the subject such as a patient to obtain the in-vivo image in a body cavity. A user such as a doctor observes the obtained in-vivo image, thereby examining whether there is a bleeding site or a tumor site being sites to be detected. The surface layer observation endoscope 6 and the surface layer observation controller 7 constitute an endoscope device for surface layer observation.

The live observation endoscope 2 is provided with a flexible elongated insertion portion 21, an operating unit 22 connected to a proximal end side of the insertion portion 21 which accepts an input of various operation signals, and a universal code 23 extending in a direction different from a direction in which the insertion portion 21 extends from the operating unit 22 in which various cables connected to the light source unit 3 and the processor 4 are embedded.

The insertion portion 21 includes a distal end portion 24 in which an image sensor 202 including pixels which receive the light (photo diodes) arranged in a lattice (matrix) pattern which generates an image signal by performing photoelectric conversion on the light received by the pixels is embedded, a bending portion 25 which is bendable and formed of a plurality of bending pieces, and an elongated flexible tube portion 26 with flexibility connected to a proximal end side of the bending portion 25.

The operating unit 22 includes: a bending nob 221 configured to bend the bending portion 25 in vertical and horizontal directions; a treatment tool insertion portion 222 through which the surface layer observation endoscope 6 and treatment tools such as biopsy forceps, an electric scalpel, and an examination probe are configured to be inserted into the subject; and a-plurality of switches 223 configured to input an instruction signal for allowing the light source unit 3 to emit the illumination light, an operation instruction signal of the treatment tool and an external device connected to the processor 4, a water delivery instruction signal for delivering water, a suction instruction signal for performing suction and the like. The surface layer observation endoscope 6 and the treatment tool inserted through the treatment tool insertion portion 222 are exposed from an aperture (not illustrated) through a treatment tool channel (not illustrated) provided on a distal end of the distal end portion 24.

The universal code 23 at least includes a light guide 203 and a cable assembly formed of one signal line or a plurality of assembled signal lines embedded therein. The cable assembly being a signal line configured to transmit and receive the signal between the live observation endoscope 2 and the light source unit 3 and processor 4 includes the signal line for transmitting and receiving setting data, the signal line for transmitting and receiving the image signal, the signal line for transmitting and receiving a driving timing signal for driving the image sensor 202 and the like.

The live observation endoscope 2 is provided with an imaging optical system 201, the image sensor 202, the light guide 203, an illumination lens 204, an A/D converter 205, and an imaging information storage unit 206.

The imaging optical system 201 is provided on the distal end portion 24 and collects at least the light from the observed region. The imaging optical system 201 is formed of one or a plurality of lenses. The imaging optical system 201 may also be provided with an optical zooming mechanism which changes an angle of view and a focusing mechanism which changes a focal point.

The image sensor 202 is provided so as to be perpendicular to an optical axis of the imaging optical system 201 and performs the photoelectric conversion on an image of the light formed by the imaging optical system 201 to generate the electric signal (image signal). The image sensor 202 is realized by using a charge coupled device (CCD) image sensor, a complementary metal oxide semiconductor (CMOS) image sensor or the like.

The image sensor 202 includes a plurality of pixels which receives the light from the imaging optical system 201 arranged in a lattice (matrix) pattern. The image sensor 202 performs the photoelectric conversion on the light received by each pixel to generate the electric signal (also referred to as the image signal and the like). The electric signal includes a pixel value (luminance value) of each pixel, positional information of the pixel and the like.

The light guide 203 is formed of a glass fiber and the like and serves as a light guide path of the light emitted from the light source unit 3.

The illumination lens 204 is provided on a distal end of the light guide 203 and diffuses the light guided by the light guide 203 to emit from the distal end portion 24 to outside.

The A/D converter 205 A/D converts the electric signal generated by the image sensor 202 and outputs the converted electric signal to the processor 4.

The imaging information storage unit 206 stores data including various programs for operating the live observation endoscope 2, various parameters required for the operation of the live observation endoscope 2, identification information of the live observation endoscope 2 and the like.

Next, a configuration of the light source unit 3 will be described. The light source unit 3 is provided with an illuminating unit 31 and an illumination controller 32.

The illuminating unit 31 switches between a plurality types of illumination light of different wavelength bands to emit under the control of the illumination controller 32. The illuminating unit 31 includes a light source 31a, a light source driver 31b, and a condenser lens 31c.

The light source 31a is configured to emit white illumination light under the control of the illumination controller 32. The white illumination light emitted by the light source 31a is emitted from the distal end portion 24 to outside through the condenser lens 31c and the light guide 203. The light source 31a is realized by using a light source which emits the white light such as a white LED and a xenon lamp.

The light source driver 31b is configured to supply the light source 31a with current to allow the light source 31a to emit the white illumination light under the control of the illumination controller 32.

The condenser lens 31c is configured to collect the white illumination light emitted by the light source 31a to emit out of the light source unit (light guide 203).

The illumination controller 32 is configured to control the illumination light emitted by the illuminating unit 31 by controlling the light source driver 31b to turn on/off the light source 31a.

Next, a configuration of the processor 4 will be described. The processor 4 is provided with an image processing unit 41, an input unit 42, a storage unit 43, and a central controller 44.

The image processing unit 41 is configured to execute predetermined image processing based on the electric signal output from the live observation endoscope 2 (A/D converter 205) or the surface layer observation controller 7 (signal processing unit 72) to generate image information to be displayed on the display unit 5.

The input unit 42 is an interface for inputting to the processor 4 by the user and includes a power switch for turning on/off power, a mode switching button for switching among a shooting mode and various other modes, an illumination light switching button for switching the illumination light of the light source unit 3 and the like.

The storage unit 43 records data including various programs for operating the endoscope system 1, various parameters required for the operation of the endoscope system 1 and the like. The storage unit 43 is realized by using a semiconductor memory such as a flash memory and a dynamic random access memory (DRAM).

The central controller 44 is formed of a CPU and the like and is configured to perform driving control of each element of the endoscope system 1, input/output control of information to/from each element and the like. The central controller 44 transmits the setting data (for example, the pixel to be read) for imaging control recorded in the storage unit 43, a timing signal regarding imaging timing and the like to the live observation endoscope 2 and the surface layer observation controller 7 through a predetermined signal line.

Next, the display unit 5 will be described. The display unit 5 receives a display image signal generated by the processor 4 through a video cable to display the in-vivo image corresponding to the display image signal. The display unit 5 is formed of a liquid crystal, an organic EL (electro luminescence) or the like.

A configuration of the surface layer observation endoscope 6 will be described next. The surface layer observation endoscope 6 is provided with a flexible elongated insertion portion 61. The insertion portion 61 connected to the surface layer observation controller 7 on a proximal end side thereof has a distal end inserted into the treatment tool insertion portion 222 to extend from the distal end portion 24. The surface layer observation endoscope 6 allows a distal end face thereof to abut on the surface layer of the living body (for example, a surface of an organ) to obtain the in-vivo image (hereinafter, also referred to as surface layer image). The surface layer observation endoscope 6 allows the distal end face thereof to abut on the gland duct to obtain an image of the surface of the organ, for example. In contrast to the live observation endoscope 2 which obtains the in-vivo image obtained by capturing an image of an entire body cavity, the surface layer observation endoscope 6 obtains the surface layer image being the image of the surface layer (or to a depth of 1000 μm of the surface layer) on the surface of the organ.

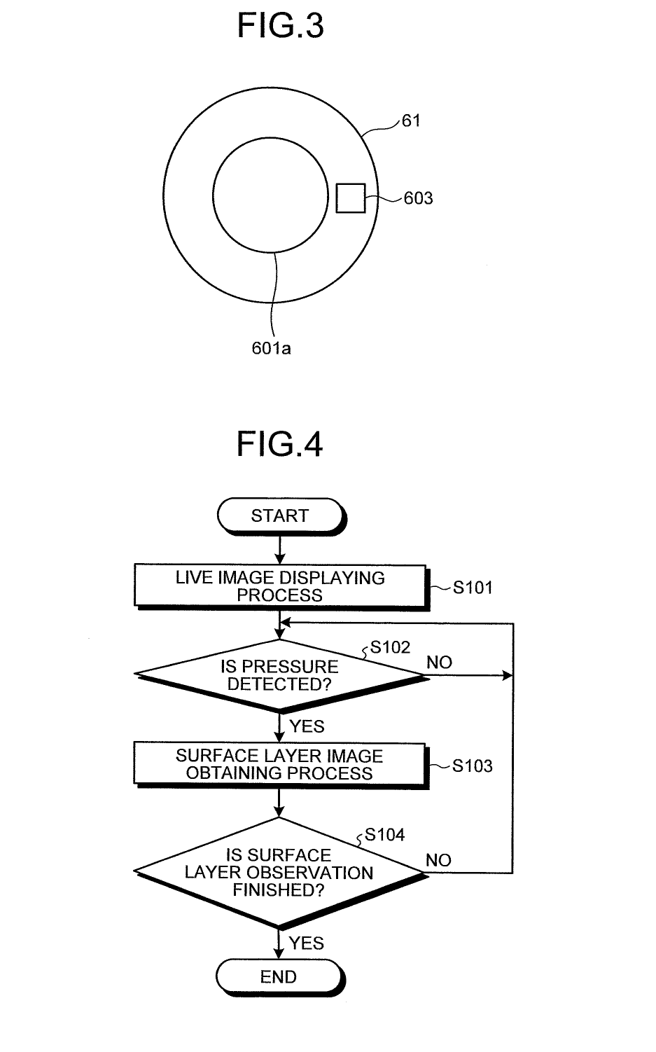

FIG. 3 is a schematic diagram illustrating a configuration of the distal end face of the surface layer observation endoscope according to the first embodiment. The insertion portion 61 is provided with an imaging optical system 601, an image sensor 602 (imaging unit), and a pressure sensor 603 (pressure detecting unit).

The imaging optical system 601 is provided on the distal end of the insertion portion 61 and is configured to collect the light at least from the observed region. The imaging optical system 601 is formed of one or a plurality of lenses (for example, a lens 601a provided on the distal end face of the insertion portion 61).

The image sensor 602 is provided so as to be perpendicular to an optical axis of the imaging optical system 601 and is configured to perform the photoelectric conversion on an image of the light formed by the imaging optical system 601 to generate the electric signal (image signal). The image sensor 602 is realized by using a CCD image sensor, a CMOS image sensor and the like.

The pressure sensor 603 is provided on the distal end face (a surface to be in contact with the surface layer of the living body) of the insertion portion 61, and is configured to convert an applied load into the electric signal to output the electric signal to the surface layer observation controller 7 (measuring unit 71) as a detection result. The pressure sensor 603 is realized by using a sensor which detects physical change such as displacement and stress by pressure as electric change such as a resistance value, capacitance, and a frequency.

As illustrated in FIG. 3, the pressure sensor 603 is provided around the lens 601a. Therefore, the image sensor 602 may capture an image without the pressure sensor 603 included in an angle of view of the imaging optical system. 601 (image sensor 602). A diameter of the distal end face of the insertion portion 61 is designed to be larger than a distance between the adjacent two gland ducts. Therefore, the distal end face of the insertion portion 61 is in contact with at least the two gland ducts and the contact portion also includes the pressure sensor 603.

The surface layer observation controller 7 is formed of a CPU and the like and is configured to perform driving control of each element of the surface layer observation endoscope 6, input/output control of information to/from each element and the like. The surface layer observation controller 7 includes the measuring unit 71 and the signal processing unit 72.

The measuring unit 71 is configured to measure a value of pressure applied to the distal end face of the insertion portion 61 based on the electric signal output from the pressure sensor 603.

The signal processing unit 72 is configured to execute predetermined signal processing based on the electric signal from the image sensor 602 to output the electric signal after the signal processing to the processor 4 (image processing unit 41).

The surface layer observation controller 7 is configured to perform operational control of an imaging process by the image sensor 602 according to an output of the pressure value from the measuring unit 71.

FIG. 4 is a flowchart illustrating a surface layer observing process performed by the endoscope system 1 according to the first embodiment. The central controller 44 allows the display unit 5 to display the image information generated by the image processing unit 41 based on the image signal generated by the image sensor 202 as a live image (step S101). The user such as the doctor inserts the insertion portion 21 into the body cavity and moves the distal end portion 24 (imaging optical system 201) of the insertion portion 21 to a desired position while checking the live image.

Thereafter, when the insertion portion 21 reaches a desired imaging position, the user inserts the insertion portion 61 of the surface layer observation endoscope 6 into the treatment tool insertion portion 222 to allow the distal end face of the insertion portion 61 to abut on the surface layer in the imaging position. When the measuring unit 71 receives the electric signal (detection result) from the pressure sensor 603, the measuring unit 71 measures the value of the pressure applied to the distal end face of the insertion portion 61 based on the received electric signal. The surface layer observation controller 7 determines that the pressure is detected when the pressure value is output from the measuring unit 71. On the other hand, the surface layer observation controller 7 repeatedly performs a detecting process of the pressure when the pressure value is not output from the measuring unit 71 (step S102: No).

When the pressure is detected (step S102: Yes), the surface layer observation, controller 7 performs the operational control of the imaging process by the image sensor 602 (step S103). According to this, the image sensor 602 may perform a surface layer image capturing process substantially at the same time as the detection of the pressure. The electric signal generated by the image sensor 602 is output to the signal processing unit 72 and subjected to predetermined signal processing, and thereafter output to the processor 4.

When the imaging process at step S103 is finished, the surface layer observation controller 7 determines whether to finish the surface layer observing process (step S104). The surface layer observation controller 7 determines whether to finish the surface layer observing process based on a control signal from the central controller 44; when the surface layer observation controller 7 determines to finish the process (step S104: Yes), the surface layer observing process is finished, and when the surface layer observation controller 7 determines not to finish the process (step S104: No), the surface layer observation controller 7 returns to step S102 and continues the surface layer observing process (an image obtaining process by the image sensor 602).

In this manner, in the first embodiment, it is possible to obtain the surface layer image at timing at which the distal end face of the insertion portion 61 is brought into contact with the surface layer of the living body by performing the surface layer image capturing process based on the pressure detection. Since the surface layer image is obtained at the timing at which the distal end face of the insertion portion 61 is in contact with the surface layer of the living body also when a position of the distal end face of the insertion portion 61 with respect to the surface layer changes due to pulsation of the living body in the body cavity, so that it is possible to surely obtain the in-vivo image in a contact state.

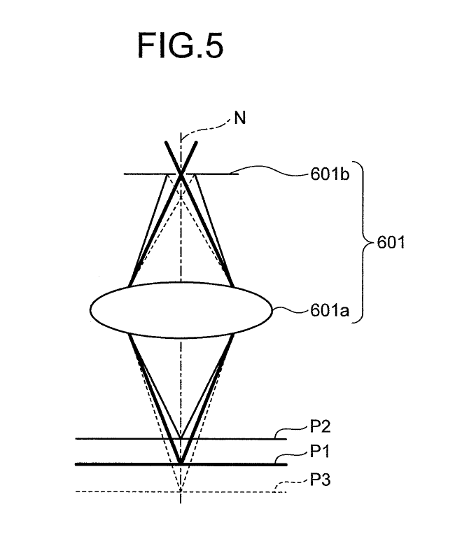

FIG. 5 is a schematic view for illustrating an example of a configuration of the distal end of the surface layer observation endoscope 6 according to the first embodiment. The imaging optical system 601 constitutes a confocal optical system by using one or a plurality of lenses (for example, the lens 601a) and a disk 601b provided in a position conjugate with a focal position of the imaging optical system 601 on which a confocal aperture such as a slit and a pinhole is formed.

The imaging optical system 601 irradiates a specimen through the slit and the pinhole on the disk 601b to pass only observation light from a cross-section (focal position) which is wanted to be observed through. That is to say, it is possible to obtain an image of each focal plane focusing on each of different focal positions P1, P2, and P3 (confocal image) by moving the imaging optical system 601 in an optical axis N direction. It is possible to obtain the confocal image when the distal end face of the insertion portion 61 is in contact with the surface layer of the living body by obtaining the confocal image at the timing of the surface layer image capturing process by the image sensor 602 described above. In this case, the imaging optical system 601 is preferably formed to be movable with respect to the distal end face of the insertion portion 61.

According to the above-described first embodiment, the surface layer image is obtained when the distal end face of the insertion portion 61 is in contact with the surface layer of the living body by performing the surface layer image capturing process based on the pressure detection, so that it is possible to surely obtain the in-vivo image while the distal end face of the insertion portion 61 is being in contact with the surface layer of the living body.

According to the above-described first embodiment, the pressure sensor 603 is provided on the distal end face of the insertion portion 61. With this structure, it is possible to obtain the actual load applied to the surface layer of the living body by the distal end face of the insertion portion 61, so that it is possible to perform observation and imaging process without damaging the living body.

### First Modification of First Embodiment

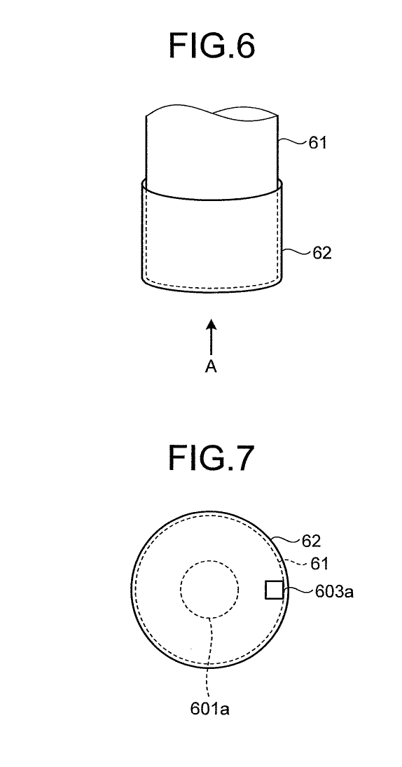

FIG. 6 is a schematic diagram illustrating a configuration of a distal end of a surface layer observation endoscope 6 according to a first modification of the first embodiment. FIG. 7 is a planar view in an arrow A direction of FIG. 6. In the above-described first embodiment, it is described that the pressure sensor 603 is provided on the distal end face of the insertion portion 61, but it is also possible to provide a pressure sensor 603a (pressure detecting unit) on a cap 62 separate from the insertion portion 61 and attach the cap 62 to the insertion portion 61 to fix. In a configuration in which the pressure sensor 603a is provided ahead of the distal end face of the insertion portion 61 also, it is possible to detect pressure in a state in which positional relationship between the cap 62 and the insertion portion 61 is fixed, so that it is possible to obtain a surface layer image at timing at which the distal end (cap 62) of the insertion portion 61 is in contact with a surface layer of a living body.

The cap 62 has a cup shape which may accommodate the distal end of the insertion portion 61 inside thereof and is provided with a pressure sensor 603a on a bottom thereof. At least the bottom of the cap 62 is formed of a light-transmissive plate-shaped member (glass or transparent resin). The pressure sensor 603a transmits an electric signal to a measuring unit 71 through a signal line not illustrated.

### Second Modification of First Embodiment

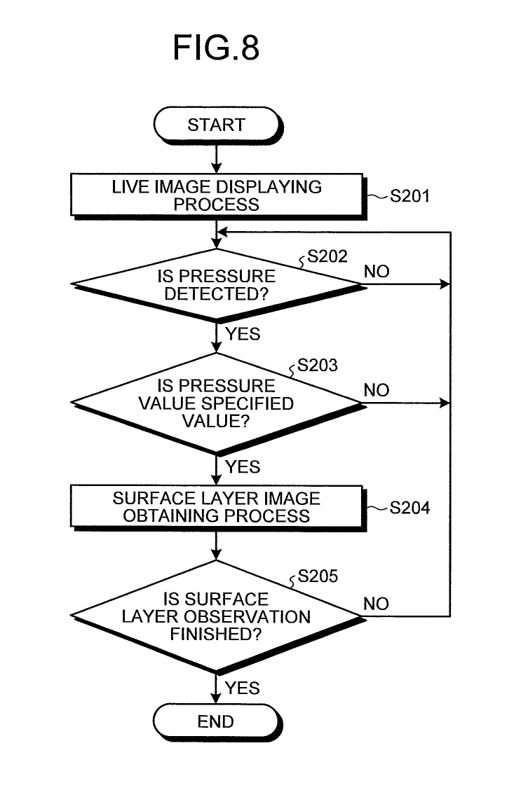

FIG. 8 is a flowchart illustrating a surface layer observing process performed by an endoscope system 1 according to a second modification of the first embodiment. Although it is described that the surface layer image capturing process is performed by the image sensor 602 when the surface layer observation controller 7 detects the pressure measured by the measuring unit 71 in the above-described first embodiment, it is also possible to provide a specified value for a pressure value and perform the surface layer image capturing process when the pressure value conforms to the specified value.

A central controller 44 is configured to cause a display unit 5 to display image information generated by an image processing unit 41 based on an image signal generated by an image sensor 202 as a live image as described above (step S201). A user such as a doctor inserts an insertion portion 21 in a desired imaging position in a body cavity and thereafter inserts an insertion portion 61 into a treatment tool insertion portion 222 to allow a distal end face of the insertion portion 61 to abut on a surface layer in the imaging position. The surface layer observation controller 7 determines that the pressure is detected when the pressure value is output from the measuring unit 71. On the other hand, the surface layer observation controller 7 repeatedly performs a detecting process of the pressure when the pressure value is not output from the measuring unit 71 (step S202: No).

When the pressure is detected (step S202: Yes), the surface layer observation controller 7 determines whether the pressure value conforms to the specified value (step S203). When the pressure value does not conform to the specified value (step S203: No), the surface layer observation controller 7 returns to step S202 to repeatedly perform the detecting process of the pressure. The surface layer observation controller 7 may obtain the specified value with reference to a storage unit 43 or obtain the specified value with reference to a storage unit provided on the surface layer observation controller 7.

On the other hand, when the pressure value conforms to the specified value (step S203: Yes), the surface layer observation controller 7 performs operational control of the imaging process by the image sensor 602 (step S204). According to this, the image sensor 602 may perform the surface layer image capturing process at substantially the same time when the insertion portion 61 presses the surface layer of a living body with predetermined pressure.

When the imaging process at step S204 is finished, the surface layer observation controller 7 determines whether to finish the surface layer observing process (step S205). The surface layer observation controller 7 determines whether to finish the surface layer observing process based on a control signal from the central controller 44; when the surface layer observation controller 7 determines finish the process (step S205: Yes), the surface layer observing process by the image sensor 602 is finished, and when the surface layer observation controller 7 determines not to finish the process (step S205: No), the surface layer observation controller 7 returns to step S202 and continues the surface layer observing process (an image obtaining process by the image sensor 602).

In this manner, in the second modification of the first embodiment, the surface layer image may be obtained when the insertion portion 61 presses the surface layer with a predetermined pressure value. It is therefore possible to obtain a plurality of surface layer images while a constant load is applied by the insertion portion 61 (i.e., while the condition of the living body is the same).

### Third Modification of First Embodiment

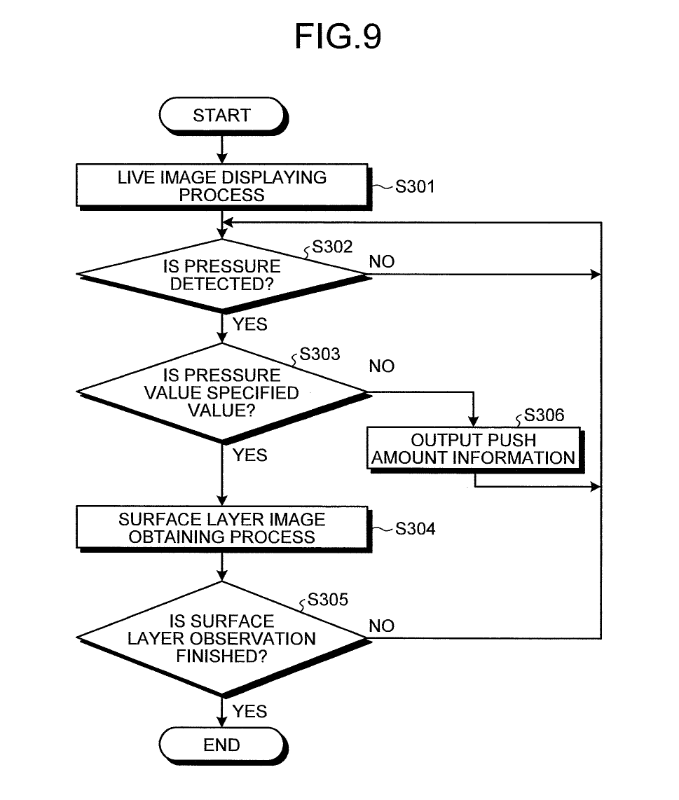

FIG. 9 is a flowchart illustrating a surface layer observing process performed by an endoscope system 1 according to a third modification of the first embodiment. Although it is described that the image sensor 602 performs the surface layer image capturing process when the surface layer observation controller 7 detects predetermined pressure in the above-described second modification of the first embodiment, it is also possible to guide a moving direction of an insertion portion 61 when a pressure value is different from a specified value.

A central controller 44 is configured to cause a display unit 5 to display image information generated by an image processing unit 41 based on an image signal generated by an image sensor 202 as a live image as described above (step S301). A user such as a doctor inserts an insertion portion 21 in a desired imaging position in a body cavity and thereafter inserts an insertion portion 61 into a treatment tool insertion portion 222 to allow a distal end face of the insertion portion 61 to abut on a surface layer in the imaging position. The surface layer observation controller 7 determines that the pressure is detected when the pressure value is output from the measuring unit 71. On the other hand, the surface layer observation controller 7 repeatedly performs a detecting process of the pressure when the pressure value is not output from the measuring unit 71 (step S302: No).

When the pressure is detected (step S302: Yes), the surface layer observation controller 7 determines whether the pressure value conforms to the specified value (step S303). When the pressure value conforms to the specified value (step S303: Yes), the surface layer observation controller 7 performs operational control of the imaging process by the image sensor 602 (step S304). According to this, the image sensor 602 can perform the surface layer image capturing process at substantially the same time when the insertion portion 61 presses the surface layer of a living body with predetermined pressure.

When the imaging process at step S304 is finished, the surface layer observation controller 7 determines whether to finish the surface layer observing process (step S305). The surface layer observation controller 7 determines whether to finish the surface layer observing process based on a control signal from the central controller 44; when the surface layer observation controller 7 determines to finish the process (step S305: Yes), the surface layer observing process by the image sensor 602 is finished, and when the surface layer observation controller 7 determines not to finish the process (step S305: No), the surface layer observation controller 7 returns to step S302 and continues the surface layer observing process (an image obtaining process by the image sensor 602).

On the other hand, when the pressure value does not conform to the specified value (step S303: No), the surface layer observation controller 7 outputs guide information for guiding the moving direction of the insertion portion 61 (step S306). Specifically, the surface layer observation controller 7 compares the pressure value with the specified value, and when the pressure value is smaller than the specified value, the surface layer observation controller 7 outputs the guide information to move the insertion portion 61 toward a surface layer side, that is to say, in a direction to push the insertion portion 61. On the other hand, the surface layer observation controller 7 outputs the guide information to move the insertion portion 61 in a direction away from the surface layer side, that is to say, to move the insertion portion 61 in a direction to draw the same from the treatment tool insertion portion 222 when the specified value is smaller than the pressure value. The surface layer observation controller 7 shifts to step S302 after outputting the guide information to repeat the pressure detecting process and subsequent processes. The guide information may be a character or an image displayed on the display unit 5 or a guide by lighting and blink of an LED and the like.

In this manner, in the third modification of the first embodiment, it is possible to obtain the surface layer image at the timing at which the surface layer is pressed with a predetermined pressure value and check the moving direction of the insertion portion 61 when the pressure value is other than the predetermined pressure value.

### Second Embodiment

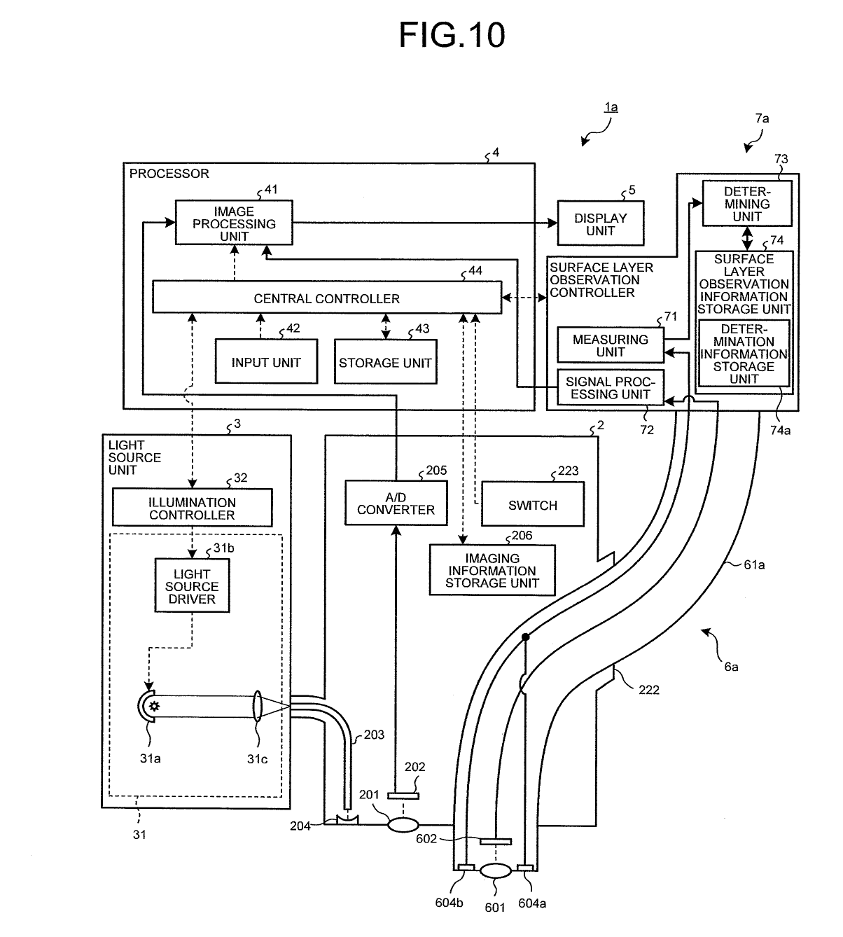

FIG. 10 is a schematic diagram illustrating a schematic configuration of an endoscope system 1a according to a second embodiment of the present invention. The same reference signs are used to designate the same elements as those of FIG. 1 and the like. Although it is described that one pressure sensor is included in the above-described first embodiment, a plurality of pressure sensors is included in the second embodiment. The endoscope system 1a according to the second embodiment is provided with a surface layer observation endoscope 6a and a surface layer observation controller 7a in place of the surface layer observation endoscope 6 and the surface layer observation controller 7 of the endoscope system 1 of the above-described first embodiment.

The surface layer observation endoscope 6a is provided with a flexible elongated insertion portion 61a. The insertion portion 61a connected to the surface layer observation controller 7a on a proximal end side thereof has a distal end inserted into a treatment tool insertion portion 222 to extend from a distal end portion 24 as is the case with the above-described insertion portion 61.

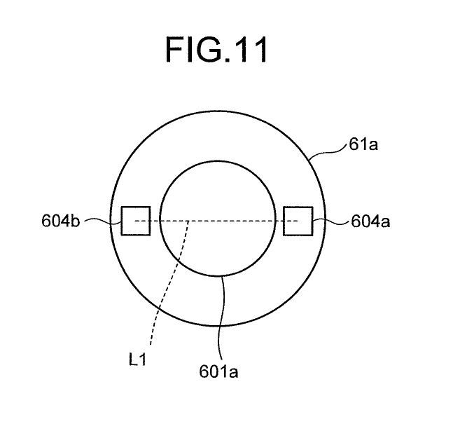

FIG. 11 is a schematic diagram illustrating a configuration of a distal end face of the surface layer observation endoscope 6a according to the second embodiment. The insertion portion 61a is provided with an imaging optical system 601, an image sensor 602, and pressure sensors 604a and 604b (pressure detecting unit).

The pressure sensors 604a and 604b are provided on the distal end face (a surface to be in contact with a surface layer of a living body) of the insertion portion 61a, and are configured to convert an applied load into electric signals to output the electric signals to the surface layer observation controller 7a (measuring unit 71). The pressure sensors 604a and 604b are realized by using sensors which detect physical change such as displacement and stress by pressure as electric change such as a resistance value, capacitance, and a frequency.

As illustrated in FIG. 11, the pressure sensors 604a and 604b are provided around a lens 601a. Therefore, the image sensor 602 may capture an image without the pressure sensors 604a and 604b included in an angle of view of the imaging optical system 601 (image sensor 602). In the second embodiment, the pressure sensors 604a and 604b are such that a line segment L1 connecting centers of the pressure sensors 604a and 604b passes through center of the lens 601a in a planar view illustrated in FIG. 11, that is to say, the pressure sensors 604a and 604b are provided in positions opposed to each other across the lens 601a. The pressure sensors 604a and 604b may be provided in any position around the lens 601a as long as they are in contact with the different gland ducts and may detect the pressure.

The surface layer observation controller 7a formed of a CPU and the like performs driving control of each element of the surface layer observation endoscope 6a, input/output control of information to/from each element and the like. The surface layer observation controller 7a includes the measuring unit 71, a signal processing unit 72, a determining unit 73, and a surface layer observation information storage unit 74.

The determining unit 73 obtains pressure values measured by the measuring unit 71 based on the electric signals generated by the pressure sensors 604a and 604b to determine whether each pressure value conforms to a specified value. The surface layer observation controller 7a performs the driving control of the surface layer observation endoscope 6a based on a determination result of the determining unit 73.

The surface layer observation information storage unit 74 records data including various programs for operating the surface layer observation controller 7a, various parameters required for the operation of the surface layer observation controller 7a and the like. The surface layer observation information storage unit 74 includes a determination information storage unit 74a which stores the pressure value for determining whether to perform an imaging process (specified value) as determination information. The specified value being the value of pressure which the insertion portion 61a applies to the surface layer of the living body is the value set as timing at which the imaging process is performed. The surface layer observation information storage unit 74 is realized by using a semiconductor memory such as a flash memory and a dynamic random access memory (DRAM).

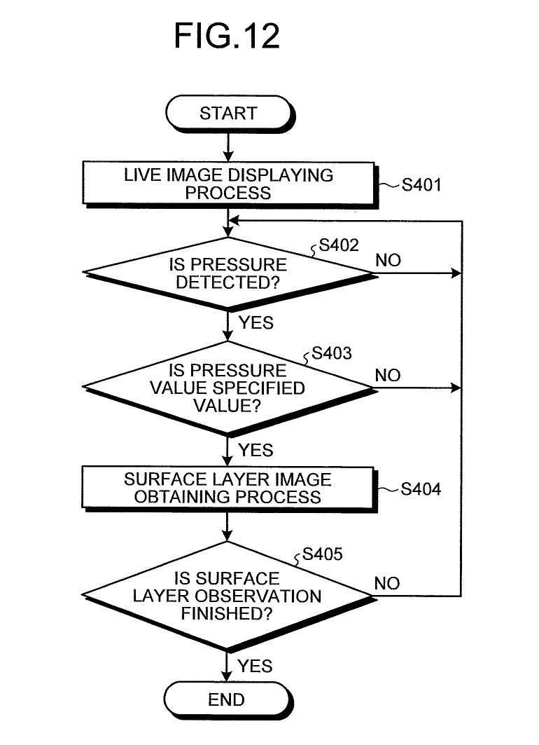

FIG. 12 is a flowchart illustrating a surface layer observing process performed by the endoscope system 1a according to the second embodiment. A central controller 44 allows a display unit 5 to display image information generated by an image processing unit 41 based on an image signal generated by an image sensor 202 as a live image (step S401). A user such as a doctor inserts an insertion portion 21 in a desired imaging position in a body cavity and thereafter inserts the insertion portion 61a into the treatment tool insertion portion 222 to allow the distal end face of the insertion portion 61a to abut on the surface layer in the imaging position while checking the live image.

The measuring unit 71 measures the pressure values applied to the distal end face of the insertion portion 61a based on the detection results of the pressure sensors 604a and 604b. The surface layer observation controller 7a determines that the pressure is detected when the pressure value is output from the measuring unit 71. On the other hand, the surface layer observation controller 7a repeatedly performs a detecting process of the pressure when the pressure value is not output from the measuring unit 71 (step S402: No).

When the surface layer observation controller 7a detects the pressure (step S402: Yes), the determining unit 73 determines whether the pressure values according to the electric signals from the pressure sensors 604a and 604b conform to the specified values with reference to the determination information storage unit 74a (step S403). Herein, when the determining unit 73 determines that at least one of the pressure values does not conform to the specified value (step S403: No), the surface layer observation controller 7a returns to step S402 to repeatedly perform the pressure detecting process.

On the other hand, when the determining unit 73 determines that the two pressure values conform to the specified values (step S403: Yes), the surface layer observation controller 7a performs operational control of the imaging process by the image sensor 602 (step S404). According to this, the image sensor 602 may perform the surface layer image capturing process at substantially the same time when the insertion portion 61a presses the surface layer of the living body with predetermined pressure.

When the imaging process at step S404 is finished, the surface layer observation controller 7a determines whether to finish the surface layer observing process (step S405). The surface layer observation controller 7a determines whether to finish the surface layer observing process based on a control signal from the central controller 44; when the surface layer observation controller 7a determines to finish the process (step S405: Yes), the surface layer observing process is finished, and when the surface layer observation controller 7a determines not to finish the process (step S405: No), the surface layer observation controller 7a returns to step S402 and continues the surface layer observing process (an image obtaining process by the image sensor 602).

In this manner, in the second embodiment, it is possible to obtain the surface layer image when the distal end face of the insertion portion 61a applies a predetermined load to the surface layer of the living body by performing the surface layer image capturing process based on the pressure detection by the two pressure sensors 604a and 604b. Furthermore, it is possible to obtain the surface layer image at timing at which orientation and an angle of the distal end face of the insertion portion 61a with respect to the surface layer of the living body are predetermined orientation and angle by using the two pressure sensors 604a and 604b. Furthermore, since the surface layer image is obtained at timing at which the distal end face of the insertion portion 61a is in contact with the surface layer of the living body in predetermined orientation also when a position of the distal end face of the insertion portion 61a with respect to the surface layer changes due to pulsation of the living body in the body cavity, so that is possible to obtain an in-vivo image at a stable angle of view.

According to the above-described second embodiment, the surface layer image is obtained when the distal end face of the insertion portion 61a is in contact with the surface layer of the living body by performing the surface layer image capturing process based on the pressure detection, so that it is possible to surely obtain the in-vivo image while the distal end face of the insertion portion 61a is being in contact with the surface layer of the living body.

According to the above-described second embodiment, the image sensor 602 performs the image obtaining process when the pressure values measured by the measuring unit 71 based on the detection results of the two pressure sensors 604a and 604b conform to the specified values, so that it is possible to obtain the surface layer image while the load of the insertion portion 61a to the surface layer of the living body is constant.

According to the above-described second embodiment, the image sensor 602 performs the image obtaining process when the pressure values measured by the measuring unit 71 based on the detection results of the two pressure sensors 604a and 604b conform to the specified values, so that it is possible to obtain the surface layer image when the orientation and the angle of the distal end face of the insertion portion 61a with respect to the surface layer of the living body are the predetermined orientation and angle (that is, condition of the living body is the same).

It is described that the image sensor 602 performs the image obtaining process when the pressure values based on the detection results of the two pressure sensors 604a and 604b conform to the specified values in the above-described second embodiment; the same specified value or different specified values may be used for the respective pressure values. It is possible to specify the orientation and the angle of the distal end face by setting the specified values for the respective pressure values. The determining unit 73 may determine with reference to the determination information in the storage unit 43 when the determination information is stored in the storage unit 43.

Although it is described that the two pressure sensors 604a and 604b are included in the above-described second embodiment, three or more pressure sensors may also be included. When the three or more pressure sensors are included, the pressure sensors are provided around the lens 601a.

### Third Embodiment

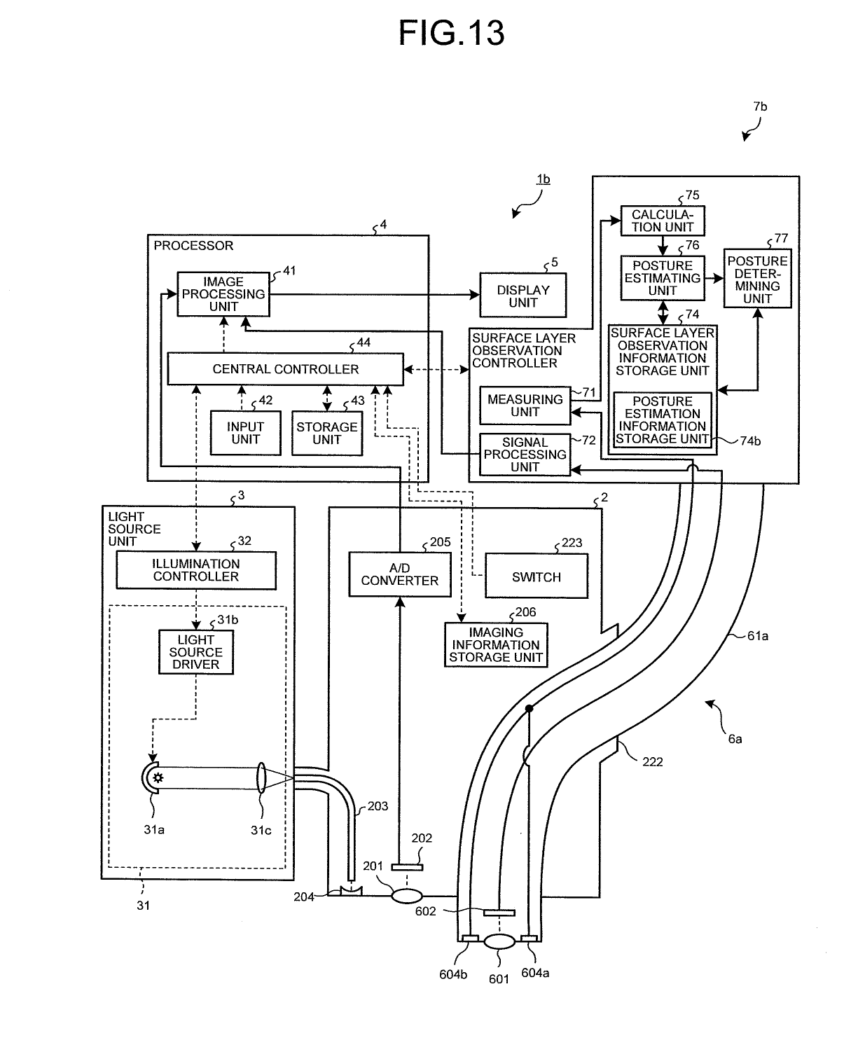

FIG. 13 is a schematic diagram illustrating a schematic configuration of an endoscope system 1b according to a third embodiment of the present invention. The same reference signs are used to designate the same elements as those of FIG. 1 and the like. Although it is described that the pressure values based on the detection results of the two pressure sensors are compared with the specified values in the above-described second embodiment, in the third embodiment, posture (orientation and an angle with respect to a surface layer) of a distal end face of an insertion portion 61a is estimated based on the pressure values based on the detection results of the two pressure sensors. The endoscope system 1b according to the third embodiment is provided with a surface layer observation controller 7b in place of the surface layer observation controller 7 of the endoscope system 1a of the above-described second embodiment.

The surface layer observation controller 7b formed of a CPU and the like performs driving control of each element of a surface layer observation endoscope 6a, input/output control of information to/from each element and the like. The surface layer observation controller 7b includes a measuring unit 71, a signal processing unit 72, a surface layer observation information storage unit 74, calculation unit 75, a posture estimating unit 76, and a posture determining unit 77.

The calculation unit 75 obtains the pressure values measured by the measuring unit 71 based on detection results of pressure sensors 604a and 604b to calculate a difference value between the pressure values. The calculation unit 75 outputs the calculated difference value to the posture estimating unit 76.

The posture estimating unit 76 estimates the posture (the orientation and the angle with respect to the surface layer) of the distal end face of the insertion portion 61a based on an arithmetic result (difference value) of the calculation unit 75.

The posture determining unit 77 determines whether the posture of the distal end face of the insertion portion 61a estimated by the posture estimating unit 76 is specified posture. The surface layer observation controller 7b performs the driving control of the surface layer observation endoscope 6a based on a determination result of the posture determining unit 77.

The surface layer observation information storage unit 74 according to the third embodiment includes a posture estimation information storage unit 74b which stores a posture estimation value for estimating the posture (the orientation and the angle with respect to the surface layer) of the distal end face of the insertion portion 61a as estimation information in place of a determination information storage unit 74a. The posture estimation value being the value set according to the difference value is the value for estimating the posture (the orientation and the angle with respect to the surface layer) of the distal end face of the insertion portion 61a therefrom.

The surface layer observation information storage unit 74 stores set specified posture (angle). The specified posture may be set through an input unit 42 or may be set through an input unit provided on the surface layer observation controller 7b. The specified posture may be set by inputting the angle or by inputting an organ to be observed and automatically setting the angle according to the input organ, for example. In this case, the surface layer observation information storage unit 74 stores a relation table in which relationship between the organ and the specified posture (angle) is stored.

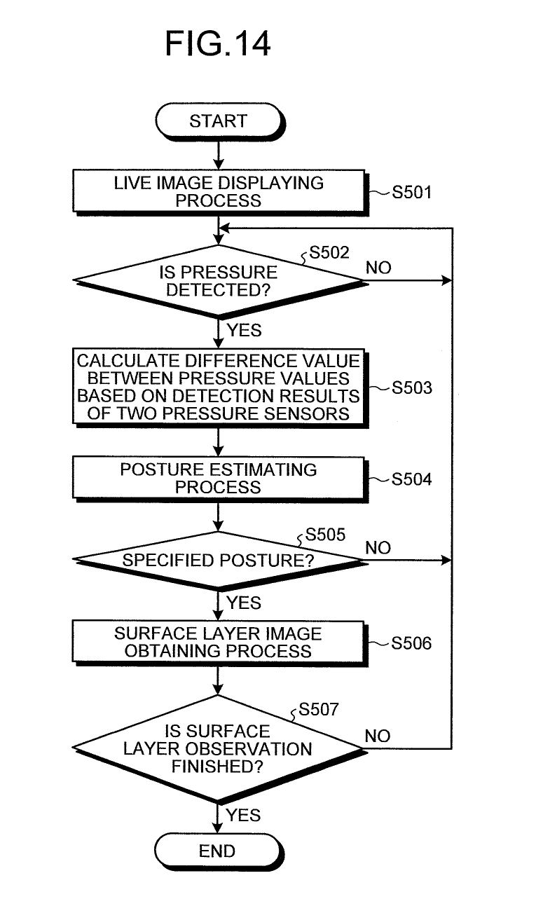

FIG. 14 is a flowchart illustrating a surface layer observing process performed by the endoscope system 1b according to the third embodiment. A central controller 44 allows a display unit 5 to display image information generated by an image processing unit 41 based on an image signal generated by an image sensor 202 as a live image (step S501). A user such as a doctor inserts an insertion portion 21 to a desired imaging position in a body cavity and thereafter inserts an insertion portion 61a into a treatment tool insertion portion 222 to allow a distal end face of the insertion portion 61a to abut on the surface layer in the imaging position while checking the live image.

The measuring unit 71 measures the pressure value applied to each sensor (the distal end face of the insertion portion 61a) based on the detection results of the pressure sensors 604a and 604b. The surface layer observation controller 7b determines that pressure is detected when the pressure value is output from the measuring unit 71. On the other hand, the surface layer observation controller 7b repeatedly performs a detecting process of the pressure when the pressure value is not output from the measuring unit 71 (step S502: No).

When the surface layer observation controller 7b detects the pressure (step S502: Yes), the calculation unit 75 calculates the difference value between the pressure values (step S503). Specifically, the calculation unit 75 calculates an absolute value of difference between the pressure values generated based on the detection results of the pressure sensors 604a and 604b as the difference value.

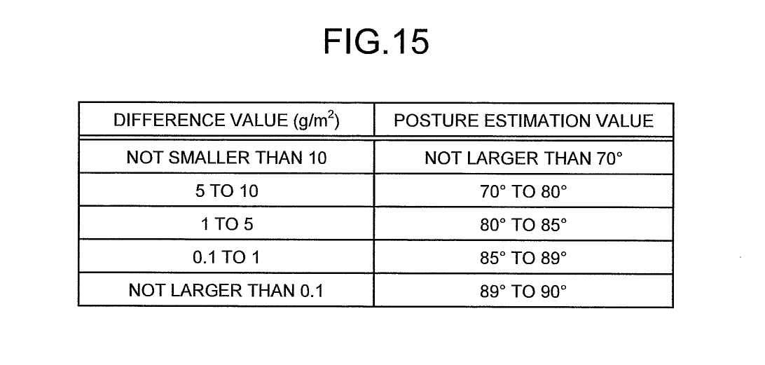

When the difference value is calculated by the calculation unit 75, the posture estimating unit 76 estimates the posture (the orientation and the angle with respect to the surface layer) of the distal end face of the insertion portion 61a from the difference value (step S504). FIG. 15 is a schematic view illustrating an example of a posture estimation table used in an image obtaining process performed by the endoscope system 1b according to the third embodiment. The posture estimation information storage unit 74b stores the posture estimation table illustrating relationship between the difference value calculated by the calculation unit 75 and the posture estimation value being a range of the angle (posture) of the distal end face of the insertion portion 61a when the surface layer of a living body is regarded to be horizontal. The posture estimating unit 76 estimates the range of the angle (posture) of the distal end face based on the difference value calculated by the calculation unit 75 with reference to the posture estimation table. For example, when the difference value obtained from the measuring unit 71 is 0.08, the posture (angle) of the distal end face is estimated to be 89 to 90 degrees with respect to the surface layer of the living body.

The posture determining unit 77 determines whether the distal end face of the insertion portion 61a is in the specified posture by determining whether the posture of the distal end face estimated by the posture estimating unit 76 is included in a range of the specified posture (step S505). Specifically, when the range of the specified posture is set to 89 to 90 degrees, the posture determining unit 77 determines that the posture is included in, the range of the specified posture if the posture of the distal end face estimated by the posture estimating unit 76 is 89 to 90 degrees.

When the posture determining unit 77 determines that the distal end face of the insertion portion 61a is in the specified posture (step S505: Yes), the surface layer observation controller 7b performs operational control of an imaging process by an image sensor 602 (step S06). According to this, the image sensor 602 may perform the surface layer image capturing process at substantially the same time when the insertion portion 61a abuts on the surface layer of the living body (gland duct) in predetermined posture. On the other hand, when the posture determining unit 77 determines that the estimated posture is not the specified posture (step S505: No), the surface layer observation controller 7b returns to step S502 to repeatedly perform the detecting process of the pressure.

When the imaging process at step S506 is finished, the surface layer observation controller 7b determines whether to finish the surface layer observing process (step S507). The surface layer observation controller 7b determines whether to finish the surface layer observing process based on a control signal from the central controller 44; when the surface layer observation controller 7b determines to finish the process (step S507: Yes), the surface layer observing process is finished, and when the surface layer observation controller 7b determines not to finish the process (step S507: No), the surface layer observation controller 7b returns to step S502 and continues the surface layer observing process (the image obtaining process by the image sensor 602).

In this manner, in the third embodiment, it is possible to obtain the surface layer image in a state in which the orientation and the angle of the distal end face of the insertion portion 61a with respect to the surface layer of the living body are specified to be predetermined orientation and angle by performing the imaging process based on the estimated posture of the distal end face. Furthermore, since the surface layer image is obtained at the timing at which the distal end face of the insertion portion 61a is in contact with the surface layer of the living body in predetermined posture also when a position of the distal end face of the insertion portion 61a with respect to the surface layer changes due to pulsation of the living body in the body cavity, it is possible to obtain an in-vivo image at a stable angle of view.

According to the above-described third embodiment, the surface layer image is obtained when the distal end face of the insertion portion 61a is in contact with the surface layer of the living body by performing the surface layer image capturing process based on the pressure detection, so that it is possible to surely obtain the in-vivo image while the distal end face of the insertion portion 61a is being in contact with the surface layer of the living body.

According to the above-described third embodiment, the image sensor 602 performs the image obtaining process based on the posture estimation value obtained from the pressure values measured by the measuring unit 71 based on the detection results of the two pressure sensors 604a and 604b, so that it is possible to obtain the surface layer image at timing at which the orientation and the angle of the distal end face of the insertion portion 61a with respect to the surface layer of the living body are the predetermined orientation and angle. Therefore, it is possible to obtain the surface layer image in which the condition of the living body is the same.

Although it is described that the posture estimation value determined according to the difference value has a predetermined angle range in the above-described third embodiment, it is also possible to set one difference value according to a predetermined angle to perform the imaging process. For example, when the specified posture (angle) is set to 90 degrees and the difference value according to the angle (for example, 0) is set, the image sensor 602 may perform the surface layer image capturing process while estimating that the posture of the distal end face is the specified posture (90 degrees) when the difference value is 0. The posture determining unit 77 may directly determine the posture from the difference value in addition to determining the specified posture based on the posture (angle range) estimated by the posture estimating unit 76.

### Fourth Embodiment

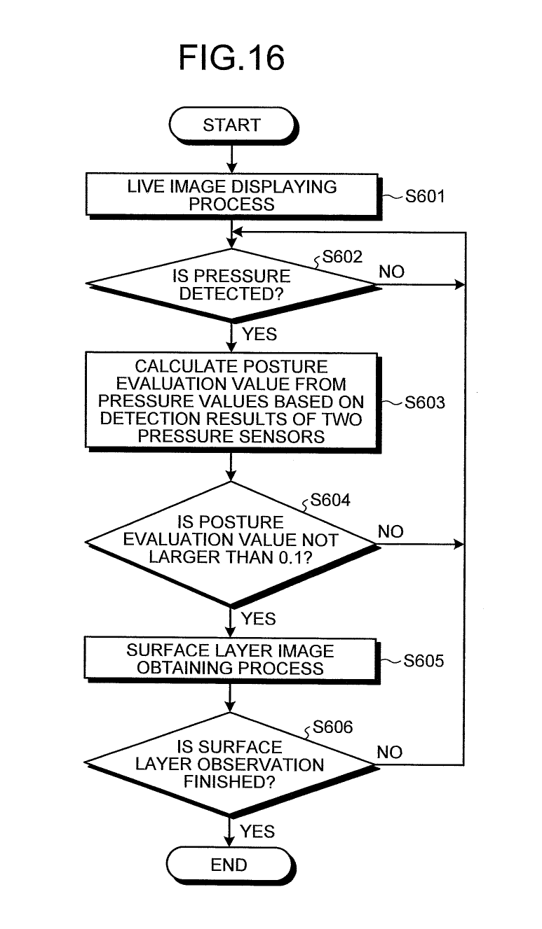

FIG. 16 is a flowchart illustrating a surface layer observing process performed by an endoscope system 1b according to a fourth embodiment. Although it is described to use the difference between the pressure values based on the detection results of the two pressure sensors in the above-described third embodiment, in the fourth embodiment, it is determined whether posture (orientation and an angle with respect to a surface layer) of a distal end face of an insertion portion 61a is specified posture based on slope obtained from the two pressure values.

A central controller 44 allows a display unit 5 to display image information generated by an image processing unit 41 based on an electric signal generated by an image sensor 202 as a live image as described above (step S601). A user such as a doctor inserts an insertion portion 21 in a desired imaging position in a body cavity and thereafter inserts the insertion portion 61a into the treatment tool insertion portion 222 to allow the distal end face of the insertion portion 61a to abut on the surface layer in the imagine position while checking the live image. The surface layer observation controller 7b determines that pressure is detected when the pressure value is output from the measuring unit 71. On the other hand, the surface layer observation controller 7a repeatedly performs a detecting process of the pressure when the pressure value is not output from the measuring unit 71 (step S602: No).

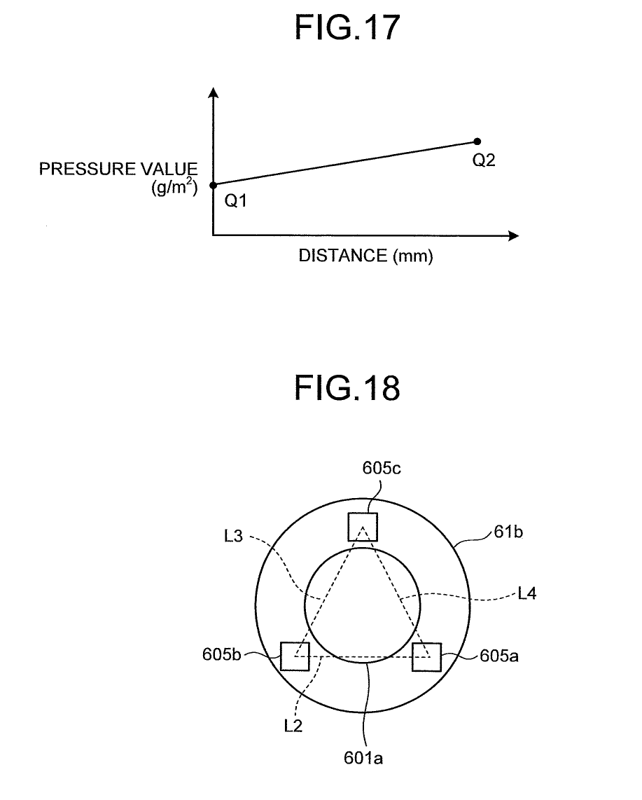

When the surface layer observation controller 7b detects the pressure (step S602: Yes), a calculation unit 75 calculates the slope being a posture evaluation value based on the two pressure values (step S603). The slope calculated by the calculation unit 75 corresponds to a slope angle of the distal end face with respect to the surface layer. FIG. 17 is a schematic view for illustrating a posture evaluation value calculating process performed by the endoscope system according to the fourth embodiment. Specifically, the calculation unit 75 plots pressure values Q1 and Q2 on a two-dimensional orthogonal coordinate system (refer to FIG. 17) having the two pressure values generated based on the pressure sensors 604a and 604b and a distance between the pressure sensors 604a and 604b as coordinate components and calculates the slope of a line segment connecting the pressure values Q1 and Q2 as the posture evaluation value. In the graph illustrated in FIG. 17, the distance between one pressure sensor and the other pressure sensor is plotted, on abscissa axis.

When the posture evaluation value is calculated by the calculation unit 75, the posture determining unit 77 determines whether the posture (the orientation and the angle with respect to the surface layer) of the distal end face of the insertion portion 61a is the specified posture from the posture evaluation value (step S604). Specifically, in a posture estimation table Illustrated in FIG. 15, the difference value and the posture evaluation value are equivalent and may be replaced with each other; when a range of the specified posture (posture estimation value) is set to 89 to 90 degrees, a range of the posture evaluation value obtained from the posture estimation value is not larger than 0.1. The posture determining unit 77 determines whether the posture (the angle with respect to the surface layer) of the distal end face of the insertion portion 61a is the specified posture by determining whether the posture evaluation value is not larger than 0.1.

Herein, when the posture determining unit 77 determines that the posture evaluation value is larger than 0.1 (not 0.1 or smaller) (step S604: No), the surface layer observation controller 7b returns to step S602 to repeatedly perform the pressure detecting process.

On the other hand, when the posture determining unit 77 determines that the posture evaluation value is not larger than 0.2 (step S604: Yes), the surface layer observation controller 7b performs operational control of an imaging process by an image sensor 602 (step S605). According to this, the image sensor 602 may perform the surface layer image capturing process at substantially the same time when the insertion portion 61a abuts on the surface layer of the living body (gland duct) in predetermined posture.

When the imaging process at step S605 is finished, the surface layer observation controller 7b determines whether to finish the surface layer observing process (step S606). The surface layer observation controller 7b determines whether to finish the surface layer observing process based on a control signal from the central controller 44; when the surface layer observation controller 7b determines to finish the process (step S606: Yes), the surface layer observing process is finished, and when the surface layer observation controller 7b determines not to finish the process (step S606: No), the surface layer observation controller 7b returns to step S602 and continues the surface layer observing process (an image obtaining process by the image sensor 602).

According to the above-described fourth embodiment, the surface layer image is obtained when the distal end face of the insertion portion 61a is in contact with the surface layer of the living body by performing the surface layer image capturing process based on the pressure detection, so that it is possible to surely obtain an in-vivo image while the distal end face of the insertion portion 61a is being in contact with the surface layer of the living body.

In the fourth embodiment, it is possible to determine the posture in consideration of the distance between the two pressure sensors 604a and 604b by making the slope of the line segment connecting the pressure values Q1 and Q2 based on the electric signals generated by the two pressure sensors 604a and 604b the posture evaluation value. Therefore, it is possible to more correctly determine the posture as compared with a case where the posture is determined only by the difference value.

### Modification of Fourth Embodiment

FIG. 18 is a schematic diagram illustrating a configuration of a distal end face of a surface layer observation endoscope according to a modification of the fourth embodiment. Although it is described that the two pressure sensors 604a and 604b are included in the above-described fourth embodiment, three or more pressure sensors may also be included. An insertion portion 61b according to the modification of the fourth embodiment includes the three pressure sensors.

In the modification of the fourth embodiment, three pressure sensors 605a, 605b, and 605c (pressure detecting units) are provided on a distal end face (a surface to be in contact with a surface layer of a living body) of the insertion portion 61b. When a load is applied to each of the pressure sensors 605a, 605b, and 605c, the pressure sensors 605a, 605b, and 605c convert the load into electric signals to output the electric signals to a surface layer observation controller 7a (measuring unit 71). The pressure sensors 605a, 605b, and 605c are realized by using sensors which detect physical change such as displacement and stress by pressure as electric change such as a resistance value, capacitance, and a frequency.

As illustrated in FIG. 18, the pressure sensors 605a, 605b, and 605c are provided around a lens 601a. In the modification of the fourth embodiment, a shape formed of line segments L2 to L4 connecting centers of the pressure sensors 605a, 605b, and 605c is an equilateral triangle in a planer view illustrated in FIG. 18. The pressure sensors 605a, 605b, and 605c may be provided on any position around the lens 601a as long as they are in contact with the different gland ducts and may detect the pressure.

When a posture estimating process is performed in accordance with steps S503 and S504 in a surface layer observing process of the above-described third embodiment (refer to FIG. 14), a calculation unit 75 calculates differences among pressure values measured based on detection results of the pressure sensors 605a, 605b, and 605c to calculate three difference values. Thereafter, a posture estimating unit 76 determines whether each difference value is included in a range of the difference value according to a posture estimation value with reference to a posture estimation table illustrated in FIG. 15 to estimate whether specified posture is realized.

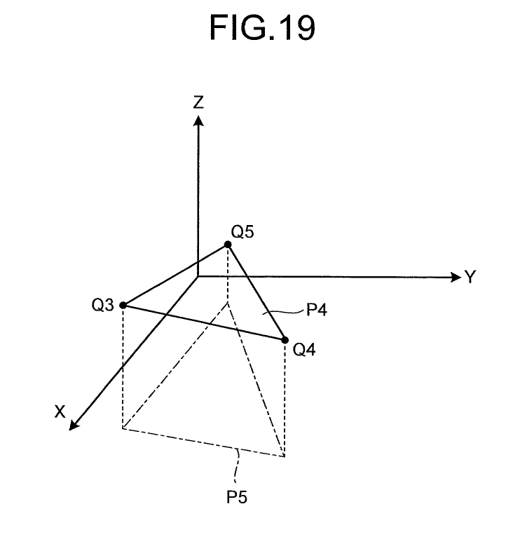

FIG. 19 is a schematic view for illustrating a posture evaluation value calculating process performed by an endoscope system 1b according to the modification of the fourth embodiment of the present invention. When a posture determining process is performed as at steps S603 and S604 of the surface layer observing process according to the above-described fourth embodiment (refer to FIG. 16), the measuring unit 71 first plots the pressure values measured based on the detection results of the pressure sensors 605a, 605b, and 605c on a three-dimensional orthogonal coordinate system (X, Y, Z) having the pressure values and positions on a plane of the pressure sensors 605a, 605b, and 605c as coordinate components. In the orthogonal coordinate system illustrated in FIG. 19, the coordinate component on the plane on which each of the pressure sensors 605a, 605b, and 605c is arranged (distal end face) is represented on an XY plane and the coordinate component of the pressure value measured based on the detection result of each pressure sensor is represented along a Z direction.

When plotting pressure values Q3, Q4, and Q5 measured by the measuring unit 71 based on the detection results of the pressure sensors 605a, 605b, and 605c, respectively, on the three-dimensional orthogonal coordinate system (X, Y, Z), a three-dimensional plane P4 is formed of line segments connecting the pressure values Q3, Q4, and Q5.

The calculation unit 75 calculates slope of the three-dimensional plane P4 with respect to a two-dimensional plane P5 by using the three-dimensional plane P4 and the two-dimensional plane having the positions of the pressure sensors 605a, 605b, and 605c on the distal end face as the coordinate components (two-dimensional plane P5) and makes the slope a posture evaluation value. Thereafter, a posture determining unit 77 determines whether the posture evaluation value is not larger than 0.1, for example, thereby determining whether the specified posture is realized.

According to the modification of the fourth embodiment described above, a surface layer image is obtained when the distal end face of the insertion portion 61b is in contact with the surface layer of the living body by performing the surface layer image capturing process based on the pressure detection even if the three pressure sensors 605a, 605b, and 605c are provided, so that it is possible to surely obtain an in-vivo image while the distal end face of the insertion portion 61b is being in contact with the surface layer of the living body.

It is also possible to estimate the posture by calculating the above-described posture evaluation value also when four or more pressure sensors are provided.

When the posture determining process is performed by using the three-dimensional orthogonal coordinate system in the above-described fourth embodiment and modification, a configuration without the posture estimating unit 76 is also possible. It is possible to appropriately change presence of the posture estimating unit 76 according to types of the posture determining process. A signal may transmitted/received between the calculation unit 75 and the posture determining unit 77 through the posture estimating unit 76 or directly transmitted/received between the calculation unit 75 and the posture determining unit 77.

### Fifth Embodiment

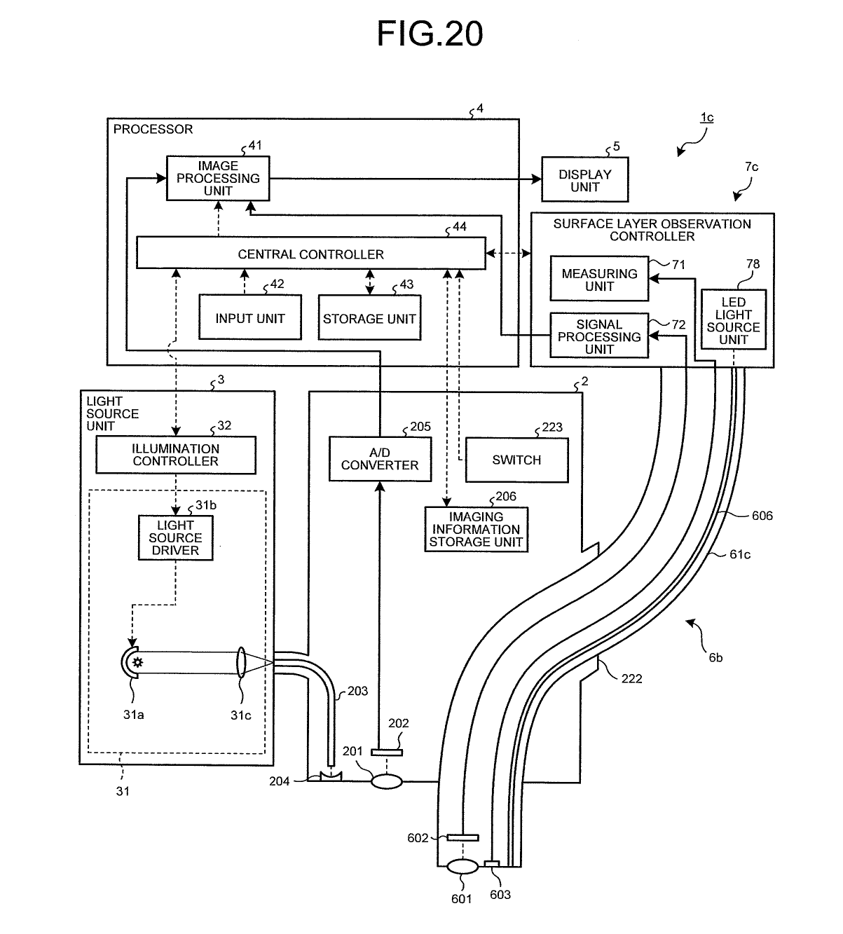

FIG. 20 is a schematic diagram illustrating a schematic configuration of an endoscope system 1c according to a fifth embodiment of the present invention. The same reference signs are used to designate the same elements as those of FIG. 1 and the like. The endoscope system 1c according to the fifth embodiment is provided with a surface layer observation endoscope 6b and a surface layer observation controller 7c in place of the surface layer observation endoscope 6 and the surface layer observation controller 7 of the endoscope system 1 of the above-described first embodiment.

The surface layer observation endoscope 6b is provided with a flexible elongated, insertion portion 61c. The insertion portion 61c connected to the surface layer observation controller 7c on a proximal end side thereof has a distal end inserted into a treatment tool insertion portion 222 to extend from a distal end portion 24 as is the case with the above-described insertion portion 61.

The insertion portion 61c is provided with an imaging optical system 601, an image sensor 602, a pressure sensor 603, and an optical irradiation fiber 606.

The optical irradiation fiber 606 realized by using an optical fiber emits illumination light incident from an LED light source unit 78 provided on the surface layer observation controller 7c outside from the distal end of the insertion portion 61c.

The surface layer observation controller 7c formed of a CPU and the like performs driving control of each element of a surface layer observation endoscope 6b, input/output control of information to/from each element and the like. The surface layer observation controller 7c includes a measuring unit 71, a signal processing unit 72, and the LED light source unit 78.

The LED light source unit 78 formed of a light emitting diode (LED) emits the illumination light generated by light emission to the optical irradiation fiber 606.

According to the above-described fifth embodiment, the illumination light is emitted from a distal end face of the insertion portion 61c by the LED light source unit 78 and the optical irradiation fiber 606, so that a further clearer in-vivo image than that of the above-described first embodiment may be obtained.

### Modification of Fifth Embodiment

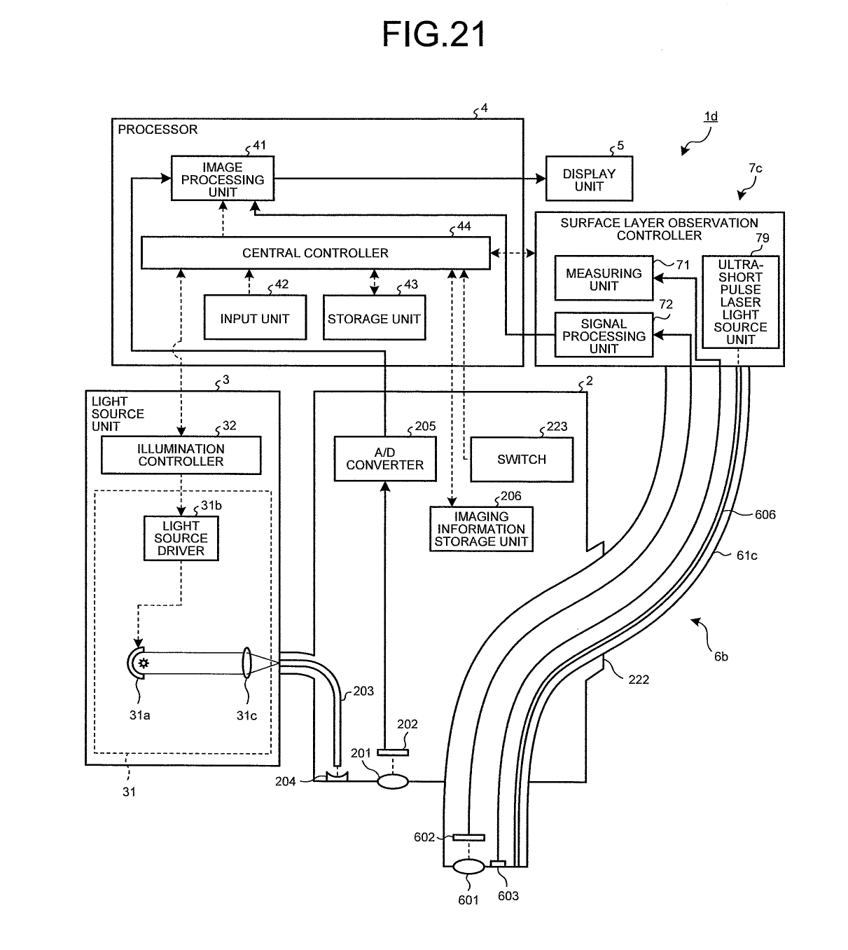

In the above-described, fifth embodiment, it is also possible to use an ultrashort pulse laser light source in place of the light emitting diode and emit ultrashort pulse laser light from ultrashort pulse laser light source to the optical irradiation fiber 606. FIG. 21 is a schematic diagram illustrating a schematic configuration of an endoscope system 1d according to a modification of the fifth embodiment of the present invention. In the endoscope system 1d according to the modification of the fifth embodiment, different from the above-described endoscope system 1c, an ultrashort pulse laser light source unit 79 including the ultrashort pulse laser light source (oscillator) is used in place of an LED light source unit 78 and a condenser lens which condenses the ultrashort pulse laser light is provided on a distal end of an insertion portion 61c as an imaging optical system 601. According to this, it becomes possible to perform two-photon excitation fluorescence observation. An ultrashort pulse laser is intended to mean a short pulse laser in which one pulse width (time width) is not longer than a femtosecond.

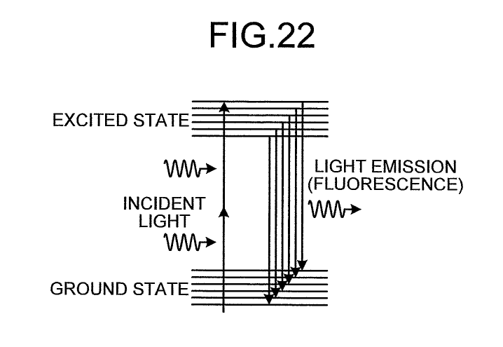

FIG. 22 is a schematic view for illustrating the two-photon excitation fluorescence observation according to the modification of the fifth embodiment. Multiphoton excitation becomes possible by using the ultrashort pulse laser light such as femtosecond laser light. For example, as illustrated in FIG. 22, when two photons simultaneously act (incident) on a molecule, the molecule transits from a ground state to an excited state and returns to the ground state while emitting light (fluorescence). Intensity of light emission (such as fluorescence) by the excitation by the two photons is proportional to a power of intensity of incident light. It becomes possible to observe a deeper portion (to a depth of several thousand μm from a wall surface (surface) of a living body, for example) of a surface layer of the living body by using this phenomenon. It is also possible to provide an optical sensor which measures the light emission intensity in place of an image sensor 602 of the insertion portion 61c.

Although the A/D converter 205 is provided on the live observation endoscope 2 in the endoscope systems 1, 1a, 1b, 1c, and 1d according to the above-described first to fifth embodiments, the A/D converter may be provided on the processor 4. In this case, the signal processing unit 72 may output an analog signal to the A/D converter provided on the processor 4.

In the endoscope system according to the above-described first to fifth embodiments, the surface layer observation endoscope 6 may be used alone as long as it is possible to check the distal end position of the insertion portion without using the live Observation endoscope 2.

According to some embodiments, it is possible to surely obtain an in-vivo image while an endoscope is being in contact with a surface layer of a living body.

The above-described first to fifth embodiments are merely examples for carrying out the present invention and the present invention is not limited to these embodiments. Various inventions may be formed by appropriately combining a plurality of elements disclosed in the embodiments and modifications of the present invention. The present invention may be variously modified according to the specification and the like and it is obvious from the above-description that various other embodiments may be made within the scope of the present invention.

As described above, the endoscope device according to some embodiments is useful for surely obtaining the in-vivo image while the endoscope is being in contact with the surface layer of the living body.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope device comprising:
an insertion portion having an imaging optical system on a distal end of the insertion portion and configured to be inserted into a living body;
a pressure detecting unit provided on the distal end of the insertion portion or ahead of the distal end and configured to detect contact with the living body by pressure; and
an imaging unit configured to image an inside of the living body through the imaging optical system when a detection result of the pressure detecting unit satisfies predetermined condition.

2. The endoscope device according to claim. 1, wherein
the imaging unit is configured to perform imaging when a pressure value measured based on the detection result of the pressure detecting unit is a predetermined value.

3. The endoscope device according to claim 1, wherein
the pressure detecting unit includes a plurality of pressure sensors.

4. The endoscope device according to claim 3, further comprising:
a posture estimating unit configured to estimate posture of the distal end with respect to the living body based on detection results of the plurality of pressure sensors; and
a posture determining unit configured to determine whether the posture is predetermined posture, wherein
the imaging unit is configured to perform imaging when the posture determining unit determines that the distal end takes the predetermined posture.

5. The endoscope device according to claim 3, wherein
the imaging unit is configured to perform imaging when each pressure value measured based on a detection result of each of the plurality of pressure sensors is a predetermined value.

6. The endoscope device according to claim 3, further comprising:
a calculation unit configured to calculate a posture evaluation value for evaluating posture of the distal end with respect to the living body in accordance with pressure value measured based on a detection result of each of the plurality of pressure sensors; and
a posture determining unit configured to determine whether the posture evaluation value calculated by the calculation unit corresponds to predetermined posture, wherein
the imaging unit is configured to perform imaging when the posture determining unit determines that the distal end takes the predetermined posture.

7. The endoscope device according to claim 6, wherein
the calculation unit is configured to calculate, as the posture evaluation value, a slope of a line segment connecting points plotted on a two-dimensional orthogonal coordinate system whose components correspond to the pressure value measured based on the detection result of each of the plurality of pressure sensors and to a distance between two of the plurality of pressure sensors.

8. The endoscope device according to claim 6, wherein
the pressure detecting unit includes three or more pressure sensors on a same plane, and
the calculation unit is configured to calculate, as the posture evaluation value, a slope of a three-dimensional plane with respect to a two-dimensional plane whose coordinate components correspond to a position of each of the three or more pressure sensors, the three-dimensional plane being formed by connecting points plotted in a three-dimensional orthogonal coordinate system whose components correspond to the pressure value measured based on the detection result of each of the three or more pressure sensors and to the position of each of the three or more pressure sensors on the same plane.

9. The endoscope device according to claim 1, wherein
the imaging optical system constitutes a confocal optical system.

10. The endoscope device according to claim 1, further comprising an ultrashort pulse laser light source unit configured to emit pulse laser light in which one pulse with is smaller than a femtosecond,
